(19) United States
(12) Patent Application Publication
(10) Pub. No.: US 2023/0067020 A1
(43) Pub. Date: Mar. 2, 2023

(54) ALCOHOL LEVEL DETECTION DEVICE

(71) Applicant: TOYODA GOSEI CO., LTD., Kiyosu-shi (JP)

(72) Inventor: Rie ONO, Kiyosu-shi (JP)

(21) Appl. No.: 17/898,999

(22) Filed: Aug. 30, 2022

(30) Foreign Application Priority Data
Sep. 2, 2021 (JP) 2021-143269

(51) Int. Cl. A61B 5/00; G01J 5/00; A61B 5/0205; A61B 5/318

(52) U.S. Cl. CPC A61B 5/4845; G01J 5/0025; A61B 5/02055; A61B 5/318; A61B 5/6893; G01J 2005/0077; A61B 5/02444

(57) ABSTRACT

An alcohol level detection device capable of detecting an alcohol level of a driver, includes a thermal camera mounted near a driver seat and to detect a body temperature of the driver, a contact electrocardiogram sensor disposed on a steering wheel steered by the driver and to detect an electrocardiogram waveform and a heart rate of the driver, a non-contact gas detection sensor disposed on a grip of the steering wheel and to detect an alcohol component contained in a gas discharged from a skin of the driver without contact with the skin, and a determination circuit which determine the alcohol level of the driver based on a measurement value of the body temperature detected by the thermal camera, measurement values of the electrocardiogram waveform and the heart rate detected by the contact electrocardiogram sensor, and a value of the alcohol component detected by the gas detection sensor.

### CROSS-REFERENCE TO RELATED APPLICATIONS

The present application claims the benefit of priority of Japanese Patent Application No. 2021-143269, filed on Sep. 2, 2021, the content of which is incorporated herein by reference.

### TECHNICAL FIELD

The present invention relates to an alcohol level detection device mounted on a vehicle and capable of detecting an alcohol level of a driver.

### BACKGROUND ART

In related art, such an alcohol level detection device includes a device that detects the alcohol level of a driver by measuring an atmospheric alcohol concentration of a driver seat in which the driver is seated and the body temperature and the heart rate of the driver (see, for example, JP-A-2019-91232). In addition, the alcohol level detection device includes a device configured to detect the alcohol level of a person by detecting a concentration of an alcohol component contained in the sweat of the person (see JP-A-2009-73422).

The alcohol level detection device disclosed in JP-A-2019-91232 is configured to measure the body temperature and the heart rate of the driver, in which, however, the alcohol component is detected by the measurement of the atmospheric alcohol concentration of the driver seat. Therefore, an alcohol component that is not discharged from the driver, for example, one generated by an alcohol sterilization spray, an aromatic, or the like, or an alcohol component of an occupant in a passenger seat passenger may also be detected, which may be erroneously detected as the alcohol level of the driver. In addition, the alcohol level detection device described in JP-A-2009-73422 is configured to detect the alcohol level of the person holding the steering wheel by detecting the concentration of the alcohol component contained in the sweat of the person, and is configured to be able to detect the alcohol component discharged from the driver. This device perspires the driver by a perspiration acceleration unit to detect the alcohol component concentration in the sweat, and thus cannot quickly detect the alcohol component.

### SUMMARY OF INVENTION

The present disclosure has been made to solve the above-described problem, and an object of the present disclosure is to provide an alcohol level detection device capable of limiting erroneous detection to accurately detect an alcohol level of a driver.

An alcohol level detection device according to the present disclosure configured to be mounted on a vehicle and to detect an alcohol level of a driver, the alcohol level detection device includes:

a thermal camera configured to be mounted near a driver seat and to detect a body temperature of the driver;

a contact electrocardiogram sensor configured to be disposed on a steering wheel steered by the driver and to detect an electrocardiogram waveform and a heart rate of the driver;

a non-contact gas detection sensor configured to be disposed on a grip of the steering wheel gripped by the driver and to detect an alcohol component contained in a gas discharged from a skin of the driver without contact with the skin; and

a determination circuit configured to determine the alcohol level of the driver based on a measurement value of the body temperature detected by the thermal camera, measurement values of the electrocardiogram waveform and the heart rate detected by the contact electrocardiogram sensor, and a value of the alcohol component detected by the gas detection sensor.

According to the alcohol level detection device of the present disclosure, since the alcohol component contained in the gas discharged from the hand of the driver gripping the grip of the steering wheel is detected by the gas detection sensor capable of detecting the alcohol component without contact with the skin, the alcohol component discharged from the driver can be detected accurately. In addition, the alcohol level detection device of the present disclosure is also configured to detect the body temperature, the electrocardiogram waveform, the heart rate, and the like of the driver by the thermal camera and the contact electrocardiogram sensor, and thus can prevent the occurrence of erroneous detection due to spoofing, and can also detect a change in the state of the driver due to alcohol by measuring the amount of change in the body temperature, the electrocardiogram waveform, and the heart rate of the driver.

Therefore, according to the alcohol level detection device of the present disclosure, it is possible to accurately detect the alcohol level of the driver while limiting erroneous detection.

In the alcohol level detection device according to the present disclosure, it is preferable that the gas detection sensor is disposed in a recess formed by recessing the grip from a surface. According to the above configuration, the gas detection sensor can be stabled in a state without contact with the hand of the driver, and since the gas discharged from the skin of the driver can be stored in the recess, the alcohol component in the gas can be detected more reliably.

Specifically, semiconductor type gas detection sensor can be used as an example.

Moreover, in the alcohol level detection device according to the present disclosure, it is preferable that the gas detection sensor and the contact electrocardiogram sensor are disposed in a region that can be simultaneously covered by a hand of the driver when the driver grips the grip. According to the above configuration, it is possible to measure the body temperature, the electrocardiogram waveform, and the heart rate of the driver simultaneously with the detection of the alcohol component.

Moreover, in the alcohol level detection device according to the present disclosure, it is preferable that the contact electrocardiogram sensor also functions as a touch sensor for detecting whether the driver is gripping. According to the above configuration, since the contact electrocardiogram sensors can also detect the gripping state of the steering wheel of the driver, in the case of, for example, being mounted on a vehicle having an automatic driving function, it is not necessary to separately dispose a gripping detection sensor on the steering wheel for detecting the gripping state of the steering wheel by the driver at the time of switching from automatic driving to manual driving.

Moreover, the alcohol level detection device according to the present disclosure is preferably further includes at least one of a camera configured to capture an image of the driver, a pulse wave sensor configured to detect a pulse wave or a blood oxygen concentration of the driver, or a posture detection sensor configured to detect a posture of the driver. According to the above configuration, for example, the change in the state of the driver such as the occurrence of sleepiness can be detected in more detail by detecting the change in the eyelids of the driver by the camera, detecting the change in the pulse wave or the blood oxygen concentration of the driver by the pulse wave sensors, and detecting the change in the posture of the driver by the posture detection sensor.

### BRIEF DESCRIPTION OF DRAWINGS

FIG. 1 is a schematic diagram showing an alcohol level detection device according to an embodiment of the present invention.

FIG. 2 is a schematic plan view showing a steering wheel constituting the alcohol level detection device of the embodiment.

FIG. 3A is a partially enlarged plan view showing a portion of a gas detection sensor disposed on the steering wheel in the alcohol level detection device of the embodiment.

FIG. 3B is a cross-sectional view of a gas detection sensor disposed on the steering wheel in the alcohol level detection device of the embodiment.

FIG. 4 is a block diagram illustrating a determination circuit in the alcohol level detection device according to the embodiment.

### DESCRIPTION OF EMBODIMENTS

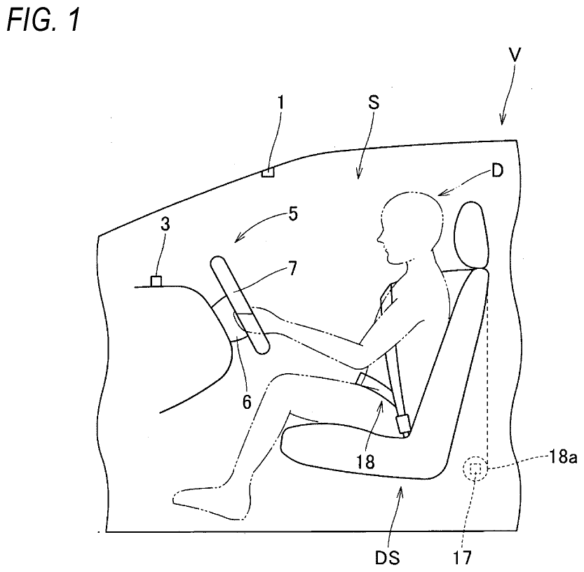

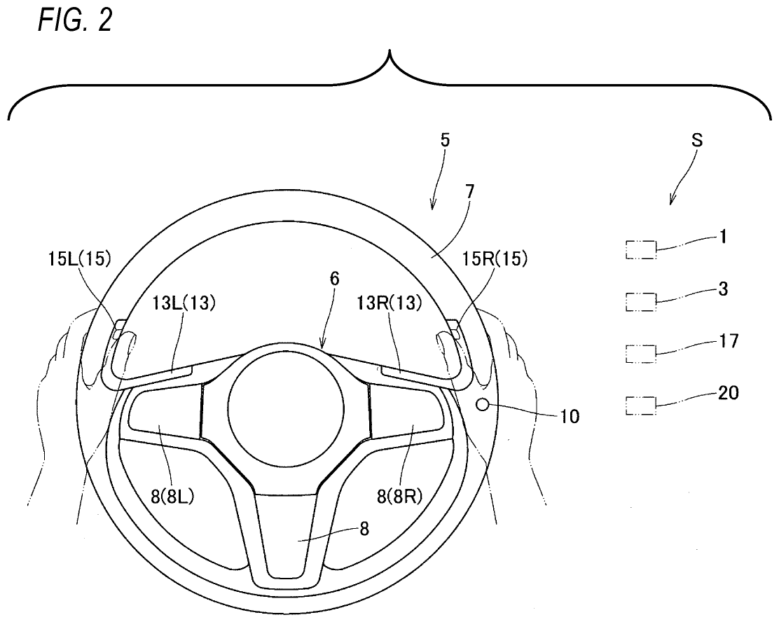

Hereinafter, an embodiment of the present disclosure will be described with reference to the drawings. As shown in FIGS. 1 and 2, an alcohol level detection device S of the embodiment is configured to include, in a vehicle V: a thermal camera 1 mounted near a driver seat DS; a camera 3 capable of capturing an image of a driver D seated in the driver seat DS; a steering wheel 5 steered by the driver D; a gas detection sensor 10, contact electrocardiogram sensors 13, and pulse wave sensors 15, each provided on the steering wheel 5; a posture detection sensor 17 disposed on a side closer to the driver seat DS; and a determination circuit 20 capable of determining an alcohol level of the driver D.

The thermal camera 1 is mounted in the vicinity of the driver seat DS and is capable of detecting the body temperature of the driver D seated in the driver seat DS. Therefore, a thermal camera mounted on the vehicle for air conditioner control can be used directly. The thermal camera 1 is set to constantly detect the body temperature of the driver D during driving (when the engine is driven). In the case of the embodiment, as shown in FIG. 1, the thermal camera 1 is mounted on a portion of an overhead console (specifically, illumination provided in an overhead console (not shown)) in front of and above the driver seat DS.

Specifically, the camera 3 capable of imaging the driver D is capable of imaging the face of the driver D at the time of driving. In the case of the embodiment, as shown in FIG. 1, an in-vehicle imaging camera of a drive recorder installed in front of the driver seat DS (specifically, above the upper surface of the instrument panel) is used directly. By using the camera 3, the state of the driver D is also monitored while performing in-vehicle imaging. The camera 3 is also set to constantly image the state of the face of the driver D at the time of driving (when the engine is driven).

As shown in FIG. 2, the steering wheel 5 steered by the driver D includes: a boss 6 connected to a rotating shaft (not shown); a substantially annular ring 7 as a grip disposed around the boss 6 and gripped during steering; and a plurality of (three in the case of the embodiment) spokes 8 connecting the boss 6 and the ring 7.

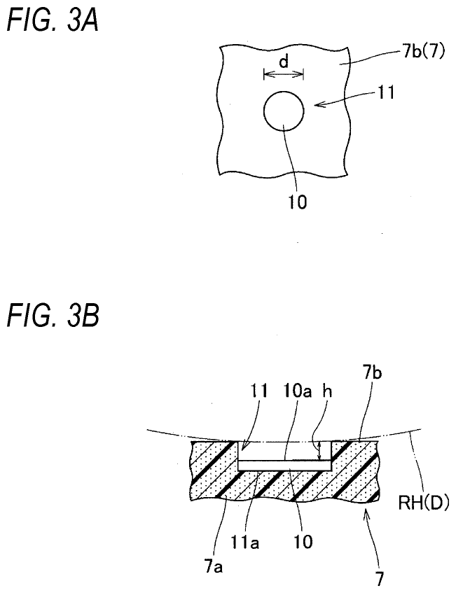

The gas detection sensor 10 can detect an alcohol component discharged from the skin of the driver D without contact with the skin, and is disposed on the ring 7 as the grip gripped by the driver D in the steering wheel 5. The gas detection sensor 10 is configured to be disposed in a region of the ring 7 that is normally gripped by the driver D during steering, and in the case of the embodiment, as illustrated in FIG. 2, is disposed at one position in a region that intersects with a spoke 8R disposed on the right side of the ring 7 (a region with which a right hand RH of the driver D is in contact). In addition, as shown in FIGS. 3A and 3B, the gas detection sensor 10 is disposed on a side closer to a bottom surface 11a of the recess 11 (tip end side of the recess) formed by partially recessing the ring 7 (specifically, a coating layer 7a constituting the surface side of the ring 7) from a surface 7b, and is thereby disposed without contact with the right hand RH (specifically, the palm) of the driver D gripping the ring 7. The gas detection sensor 10 can detect the alcohol (ethanol) component contained in the gas discharged from the palm (skin) of the right hand RH of the driver D gripping the ring 7, and is a commercially available product of a semiconductor type in the case of the embodiment. Specifically, the gas detection sensor 10 has a substantially disc shape having an outer diameter dimension d of about 7 mm (see FIGS. 3A and 3B). The recess 11 has an inner diameter set substantially equal to the outer diameter of the gas detection sensor 10, and a separation distance h between a surface 10a of the gas detection sensor 10 disposed on a side closer to the bottom surface 11a and the surface 7b of the ring 7 is set to about 2 mm (see FIGS. 3A and 3B). The recess 11 is filled with the gas discharged from the right hand RH of the driver D after about 15 s from the start of the contact with the right hand RH (palm), and the gas detection sensor 10 detects the presence or absence of alcohol (ethanol) component in the gas filled in the recess 11. In the embodiment, a sensor of the semiconductor type is used as the gas detection sensor, but a sensor of a fuel cell type, a non-diffusive infrared ray absorption type, or the like can also be used. The semiconductor type, however, is preferable in consideration of the size, cost, response performance, and the like.

The contact electrocardiogram sensors 13 are capable of detecting the electrocardiogram waveform and the heart rate of the driver D, and are disposed in region of the steering wheel 5 that are normally gripped by the driver D during steering. In the case of the embodiment, the contact electrocardiogram sensors 13 are disposed with the surfaces thereof exposed, at two positions on the steering wheel 5 that are regions to be respectively gripped by the left hand LH and the right hand RH of the driver D at the time of steering. Specifically, as shown in FIG. 2, in the steering wheel 5 during straight traveling steering, the contact electrocardiogram sensors 13 (13L, 13R) are continuously disposed to extend respectively from the inner edge sides of the intersection portions (corner portion) between the left and right spokes 8L, 8R and the ring 7 to the front edge sides of the spokes 8L, 8R and the inner edge side of the ring 7. That is, the contact electrocardiogram sensors 13 (13L, 13R) are normally disposed on the front edge sides (inner edge sides) of the regions of the corner portions to be hooked by the thumbs of the left hand LH and the right hand RH of the driver D when gripping the ring 7, and are brought into contact with the thumbs (the tips of the index fingers when gripping slightly). The right contact electrocardiogram sensor 13R is disposed in a region that can be covered simultaneously with the gas detection sensor 10 by the right hand RH of the driver D when the driver D grips the ring 7. Specifically, in a state in which the thumb of the right hand RH on the corner portion is hooked between the spoke 8R and the ring 7 and is brought into contact with the contact electrocardiogram sensor 13R, the thenar of the right hand RH covers the gas detection sensor 10. The contact electrocardiogram sensors 13L, 13R detect the electrocardiogram waveform and the heart rate of the driver D when the ring 7 is gripped by the left hand LH and the right hand RH, and is set to normally measure the electrocardiogram waveform and the heart rate when the driver D steers the steering wheel 5 with both hands in the case of the embodiment. In addition, in the case of the embodiment, the contact electrocardiogram sensors 13L, 13R are disposed to also function as a touch sensor for detecting gripped or not by the driver D. As the contact electrocardiogram sensors 13, a commercially available product is used.

The pulse wave sensors 15 are capable of detecting the pulse wave and the blood oxygen concentration of the driver D, and are disposed in regions normally gripped by the driver D during steering. In the case of the embodiment, the pulse wave sensors 15 are configured to be disposed at two positions on the ring 7 of the steering wheel 5 that are regions to be respectively in contact with the left hand LH and the right hand RH of the driver D. Specifically, as shown in FIG. 2, the pulse wave sensors 15L and 15R are disposed on the inner edge side of the ring 7, and are disposed in regions in the vicinity of the front ends in the regions for arranging the contact electrocardiogram sensors 13L, 13R, which are regions to be in contact with the fingers of the left hand LH and the right hand RH (specifically, the tips of the thumbs or the index fingers) when the driver D grips the ring 7. The pulse wave sensors 15 are set to constantly measure the pulse wave and the blood oxygen concentration when the driver D steers the steering wheel 5 in a state where at least one of the left hand LH or the right hand RH is in contact. As the pulse wave sensors 15, a commercially available product is used.

In the case of the embodiment, as shown in FIG. 1, the posture detection sensor 17 is configured with a non-contact MEMS sensor provided in a retractor 18a of a seat belt 18 worn by the driver D in the driver seat DS. The posture detection sensor 17 can detect the inclination (excessive withdrawal) of the shoulder belt in the seat belt 18, and can detect the posture change (excessive inclination of the upper body or the like) of the driver D by detecting the inclination of the shoulder belt.

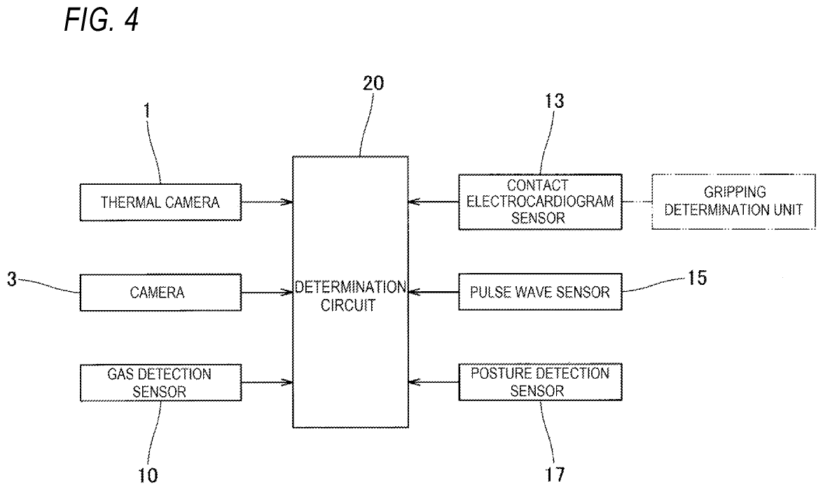

As shown in FIG. 4, the determination circuit 20 is electrically connected to the thermal camera 1, the camera 3, the gas detection sensor 10, the contact electrocardiogram sensors 13, the pulse wave sensors 15, and the posture detection sensor 17, and is set to be capable of determining the alcohol level of the driver D based on the measurement value of the body temperature detected by the thermal camera 1, the state of the face of the driver D imaged by the camera 3, the value of the alcohol component detected by the gas detection sensor 10, the measurement values of the electrocardiogram waveform and the heart rate detected by the contact electrocardiogram sensors 13, the pulse waveform and the blood oxygen concentration detected by the pulse wave sensors 15, and the posture change of the driver D detected by the posture detection sensor 17.

According to the alcohol level detection device S of the embodiment, since the alcohol component contained in the gas discharged from the hand (right hand RH) of the driver D gripping the ring 7 as the grip of the steering wheel 5 is detected by the gas detection sensor 10 capable of detecting the alcohol component without contact with the skin, the alcohol component discharged from the driver D can be detected accurately. In addition, the alcohol level detection device S of the embodiment is also configured to detect the body temperature, the electrocardiogram waveform, the heart rate, and the like of the driver D by the thermal camera 1 and the contact electrocardiogram sensors 13 (13L, 13R), and thus can prevent the occurrence of erroneous detection due to spoofing, and can also detect a change in the state of the driver D due to alcohol by measuring the change in the body temperature, the electrocardiogram waveform, and the heart rate of the driver D.

Therefore, according to the alcohol level detection device S of the embodiment, it is possible to accurately detect the alcohol level of the driver D while limiting erroneous detection.

In addition, according to the alcohol level detection device S of the embodiment, since the gas detection sensor 10 is configured to be disposed in the recess 11 formed to recess the ring 7 from the surface, the gas detection sensor 10 can be stabled in a state without contact with the hand (right hand RH) of the driver D, and since the gas discharged from the skin of the driver D can be stored in the recess 11, the alcohol component in the gas can be detected more reliably. If such a point is not taken into consideration, the gas detection sensor may be arranged not in the recess for storing the gas discharged from the skin, but at any position where the gas detection sensor does not come into contact with the skin.

Further, in the alcohol level detection device S of the embodiment, since the gas detection sensor 10 and the contact electrocardiogram sensor 13 (13R) are arranged in the region that can be simultaneously covered by the hand (right hand RH) of the driver D when the driver D grips the ring 7, it is possible to measure the body temperature, the electrocardiogram waveform, and the heart rate of the driver simultaneously with the detection of the alcohol component. If such a point is not taken into consideration, the gas detection sensor and the contact electrocardiogram sensor may be separated from each other and disposed in regions that cannot be covered simultaneously. In the embodiment, the gas detection sensor 10 is disposed at only one position in the region covered by the right hand RH, but may be disposed at two positions, one position on each of the right hand side and the left hand side.

Furthermore, according to the alcohol level detection device S of the embodiment, the contact electrocardiogram sensors 13 are configured to also function as a touch sensor. Therefore, since the contact electrocardiogram sensors 13 can also detect the gripping state of the steering wheel 5 of the driver D, in the case of, for example, being mounted on a vehicle having an automatic driving function, it is not necessary to separately dispose a gripping detection sensor on the steering wheel for detecting the gripping state of the steering wheel by the driver at the time of switching from automatic driving to manual driving. If such a point is not taken into consideration, the contact electrocardiogram sensor may be not configured to also function as the touch sensor.

Furthermore, in addition to the thermal camera 1, the gas detection sensor 10, and the contact electrocardiogram sensors 13, the alcohol level detection device S of the embodiment is configured to include the camera 3 capable of capturing an image of the driver D, the pulse wave sensors 15 capable of detecting the pulse wave or the blood oxygen concentration of the driver D, and the posture detection sensor 17 capable of detecting the posture of the driver D. Therefore, for example, the change in the state of the driver such as the occurrence of sleepiness can be detected in more detail by detecting the change in the eyelids of the driver D by the camera 3, detecting the change in the pulse wave or the blood oxygen concentration of the driver D by the pulse wave sensors 15, and detecting the change in the posture of the driver D by the posture detection sensor 17. The alcohol level detection device S is configured to include the camera 3, the pulse wave sensors 15, and the posture detection sensor 17 in the embodiment, but may also be provided with any one or two. If the points described above are not taken into consideration, the alcohol level detection device S may be provided with none of the camera, the pulse wave sensors, or the posture detection sensor.

Hereinafter, an example of the alcohol level detection of the driver D by the alcohol level detection device S of the embodiment will be described in detail.

When the physical condition of the driver D is good, the body temperature and the heart rate normally show substantially constant values, and the electrocardiogram waveform also shows a substantially constant waveform. These numerical values and waveforms are displaced due to a change in physical condition of the driver D, and thus can be used as an index of a change in physical condition. Further, it is known that when alcohol is ingested, both the body temperature and the heart rate once increase and then decrease. In addition, when alcohol is ingested, the electrocardiogram waveform also changes with a change in the heart rate. Such a decrease in the numerical value is known to occur when the driver D becomes sleepy. Therefore, according to the alcohol level detection device S of the embodiment, it is possible to determine that the driver D is sleepy when a decrease in the body temperature and the heart rate of the driver D is detected (a change in the electrocardiogram waveform due to the decrease in the heart rate is detected) in a state where a certain alcohol component value included in the gas discharged from the right hand RH is detected by the gas detection sensor 10. Further, according to the alcohol level detection device S of the embodiment, the gas detection sensor 10 detects the alcohol component contained in the gas generated from the skin of the driver D. Therefore, it is possible to prevent erroneous detection of an alcohol component when, for example, a disinfecting gel containing an alcohol component is applied to the hand.

The alcohol level detection device S according to the embodiment is for detecting the alcohol level of the driver D not in a stationary state (static state) but during driving of the vehicle V. Therefore, in order to detect a change in physical condition of the driver D driving the vehicle V (a person not in the static state), more indices for detecting the change are preferable for enabling further accuracy improvement. In addition to the thermal camera 1, the gas detection sensor 10, and the contact electrocardiogram sensors 13, the alcohol level detection device S of the embodiment is configured to include the camera 3 capable of capturing an image of the driver D, the pulse wave sensors 15 capable of detecting the pulse wave or the blood oxygen concentration of the driver D, and the posture detection sensor 17 capable of detecting the posture of the driver D. When alcohol is ingested, the pulse wave is once amplified and then attenuated, and the blood oxygen concentration also decreases. Therefore, by measuring the pulse waveform or the blood oxygen concentration, it is possible to detect a minute change in the state of the driver D. Further, according to the alcohol level detection device S of the embodiment, it is also possible to detect a change in the state of the face of the driver D captured by the camera 3 (for example, eyelids going down) and a change in the posture of the driver D by the posture detection sensor 17 (specifically, an inclination of the upper body or the like), and thus it is possible to detect the actual state of the driver D and to detect, for example, a cloudy state and a sleeping state of the driver D.

After the determination of the alcohol level of the driver D by the determination circuit 20, for example, in a case where a change in the state, the posture, or the like of the face of the driver D is not observed, a control device (not illustrated) generates a warning by light or vibration in the steering wheel to call attention. Alternatively, in a case where a change in the state or the posture of the face of the driver D is detected (in a case where a cloudy state or a sleeping state is detected), the control device takes a measure such as switching the vehicle to an autonomous driving mode and then stopping the vehicle.

What is claimed is:

1. An alcohol level detection device configured to be mounted on a vehicle and to detect an alcohol level of a driver, the alcohol level detection device comprising:
a thermal camera configured to be mounted near a driver seat and to detect a body temperature of the driver;
a contact electrocardiogram sensor configured to be disposed on a steering wheel steered by the driver and to detect an electrocardiogram waveform and a heart rate of the driver;
a non-contact gas detection sensor configured to be disposed on a grip of the steering wheel gripped by the driver and to detect an alcohol component contained in a gas discharged from a skin of the driver without contact with the skin; and
a determination circuit configured to determine the alcohol level of the driver based on a measurement value of the body temperature detected by the thermal camera, measurement values of the electrocardiogram waveform and the heart rate detected by the contact electrocardiogram sensor, and a value of the alcohol component detected by the gas detection sensor.

2. The alcohol level detection device according to claim 1, wherein
the gas detection sensor is disposed in a recess formed by recessing the grip from a surface.

3. The alcohol level detection device according to claim 1, wherein
the gas detection sensor is of a semiconductor type.

4. The alcohol level detection device according to claim 1, wherein
the gas detection sensor and the contact electrocardiogram sensor are disposed in a region that can be simultaneously covered by a hand of the driver when the driver grips the grip.

5. The alcohol level detection device according to claim 1, wherein
the contact electrocardiogram sensor also functions as a touch sensor for detecting whether the driver is gripping.

6. The alcohol level detection device according to claim 1, further comprising:
at least one of a camera configured to capture an image of the driver, a pulse wave sensor configured to detect a pulse wave or a blood oxygen concentration of the driver, or a posture detection sensor configured to detect a posture of the driver.
